(19) United States
(12) Patent Application Publication
(10) Pub. No.: US 2020/0240859 A1
(43) Pub. Date: Jul. 30, 2020

(54) HIGH-SENSITIVITY SENSOR CONTAINING LINEARLY INDUCED CRACKS AND METHOD FOR MANUFACTURING SAME

(71) Applicants: GLOBAL FRONTIER CENTER FOR MULTISCALE ENERGY SYSTEMS, Seoul (KR); SEOUL NATIONAL UNIVERSITY R&DB FOUNDATION, Seoul (KR)

(72) Inventors: Yong Whan CHOI, Seoul (KR); Taemin LEE, Gyeonggi-do (KR); Gunhee LEE, Seoul (KR); Man Soo CHOI, Seoul (KR); Daeshik KANG, Seoul (KR); Pikhitsa PETRO, Seoul (KR)

(21) Appl. No.: 15/779,202

(22) PCT Filed: Nov. 28, 2016
(86) PCT No.: PCT/KR2016/013789

§ 371 Date: May 25, 2018

(30) Foreign Application Priority Data
Nov. 30, 2015 (KR) 10-2015-0169195
Aug. 1, 2016 (KR) 10-2016-0097970

(51) Int. Cl. G01L 1/22; A61B 5/024; A61B 5/00

(52) U.S. Cl. CPC G01L 1/2287; A61B 5/6824; A61B 5/02438

(57) ABSTRACT

Provided is a high-sensitivity sensor having a conductive thin film containing linearly induced cracks. The high-sensitivity sensor relates to a sensor, obtained by forming linearly induced microcracks on a conductive thin film formed on a support, for measuring external tensile and pressure by measuring a change in the electrical resistance due to modification, short-circuiting, or openings in micro-joining structures formed by the microcracks. The high-sensitivity conductive crack sensor may be applied to high-precision measurements or artificial skins, and may be utilized as a positioning detection sensor by pixelating the sensor. Thus, the high-sensitivity sensor may be effectively used in the fields of precise measurements, bio-measurement devices through human skin, human motion measuring sensors, display panel sensors, etc.

### TECHNICAL FIELD

The present invention relates to a high-sensitivity sensor containing linearly induced cracks and a method for manufacturing the same, and more specifically relates to a high-sensitivity sensor which may be applied to metrology or artificial skin which requires a high precision to detect tensile force and pressure using a conductive thin film formed with a fine crack induced in a straight line.

### BACKGROUND ART

In general, a high-sensitivity sensor is a device that senses a minute signal and transmits it as data such as an electrical signal, which is one of the components required in the modern industry.

Among such sensors, capacitive sensors, piezoelectric sensors, strain gauges, and the like are known as sensors for measuring pressure or tensile force.

A strain gauge sensor, which is a conventional tensile sensor, is a sensor that detects a mechanical change as an electrical signal. If it is adhered to the surface of a machine or a structure, it is possible to measure a change in fine dimensions, that is, a strain, which occurs on the surface thereof, and a stress important for confirming strength and safety from the size of strain may be found.

Also, strain gauge is used to measure the deformation of the surface of the object to be measured in accordance with the change in resistance value of the metal resistance element, in general, the resistance value of a metal material increases when it is increased by external force and the resistance value of a metal material increases decreases when it is compressed. The strain gauge is also utilized as a sensing element for converting a physical quantity such as force, pressure, acceleration, displacement and torque into an electric signal, and is widely used not only for experiment and research but also for measurement control.

However, conventional strain gauge sensors are vulnerable to corrosion due to the use of metal wires, and are less sensitive, and furthermore, its output value is small, and thus additional circuitry is needed to compensate for the small signal, and semiconductor tensile sensors have heat sensitive drawbacks.

A pressure sensor is a sensor that can measure the pressure applied to the surface, which is an essential factor when making artificial skin. Strain indicates the change in horizontal length applied to the surface, while pressure indicates the force applied perpendicular to the surface.

The conventional pressure sensors measure the resistance of a thin film of silicon film, which changes with pressure, and are widely used not only for research and measurement but also for industry.

However, the conventional pressure sensors have a disadvantage in that small pressures cannot be distinguished because they are very insensitive, and they cannot be bent. These disadvantages result that the conventional pressure sensors are inapplicable to artificial skin. Therefore, it is necessary to fabricate a sensor that may bend while sensing a small pressure.

Due to the above-described problems, the sensor may be driven only in a specific environment or affected by various environmental factors, resulting in a decrease in the accuracy of measured values. In addition, there is a problem that is difficult to secure a certain measured value in repeated driving. Further, these sensors have a problem that it is difficult to manufacture a flexible structure due to a structural problem of the sensor itself.

As interest in research on the development of wearable medical and artificial electronic skin devices and high performance sensors has increased, various types of pressure sensors based on nanowires, silicon rubber, piezoelectric and organic thin film transistors that accumulate external information has been developed.

Cracks have generally been regarded as defects and have been avoided. However, recently, but studies related to cracks, cracking in thin films for nanowire production and cracks such as interconnectors, have been reported.

In addition, strain sensors based on carbon nanotubes, nanofibers, graphene platelets, and mechanical cracks have been reported.

The crack sensor was influenced by the sensory system of the spider. Spider sensory sensors are known to be very sensitive to strain and vibration.

Cracks were generally regarded as defects that should be avoided, but studies on patterning by cracks have recently reported thin film crack formation for fabrication of nanowires and interconnects, and it is reported that crack sensors similar to spider sensory systems are very sensitive to strain and vibration, but have a limit of only 2% strain.

Therefore, it is required to develop a new high sensitivity sensor which can overcome such a problem.

### DISCLOSURE

### Technical Problem

The present invention provides a high sensitivity sensor capable of detecting changes in tensile and pressure applied to various fields due to flexibility of the measurement value while maintaining the accuracy of measurement even when the influence of the environment is minimized and repeated use.

Another object of the present invention is to provide a method of manufacturing the high sensitivity sensor.

### Technical Solution

In order to solve the above technical problem, the present invention provides a high sensitivity sensor comprising:

a flexible support having a hole pattern formed therein; And

a conductive thin film formed on at least one surface of the support,

wherein the conductive thin film includes a linearly induced crack having a crack plane contacting at least a part of the surfaces and facing each other,

wherein the crack plane is linearly induced by a regular hole pattern formed on the flexible support,

wherein an external stimulus is measured by measurement of an electrical change caused by a change in contact area or a short circuit or re-contact while the crack plane moves according to an external physical stimulus.

Also, the present invention provides a manufacturing method of the high sensitivity sensor:

forming a regular hole pattern in the flexible support;

forming a conductive thin film on at least one surface of the flexible support; and

pulling the conductive thin film to induce a crack on a straight line.

### Advantageous Effects

The high sensitivity sensor of the present invention can measure tensile and/or pressure with high sensitivity by using a conductive thin film formed with a crack induced in a straight line on one surface of a support, and has flexibility and is applicable to various fields. Such a high sensitivity sensor as described above may be applied to highly precise measurement or artificial skin, and may be utilized as a positioning detection sensor by pixelizing the sensor. Thus, it is possible to provide a device for precision measurement, a motion sensor of a motion sensor, a display panel sensor, and the like.

In addition, the high-sensitivity sensor may be mass-produced by a simple process and thus has a very high economic efficiency.

### DESCRIPTION OF DRAWINGS

FIG. 1 is a model of a crack lip having a grain size of 1.

FIG. 2 shows a portion of a complex plane surrounded by an integrated contour.

FIG. 3 is a schematic view of a manufacturing process of a crack sensor according to an embodiment.

FIG. 4 is a SEM image (c, d) showing changes in the sensor surface (a, b) and cracks on the conductive thin film before and after pulling the crack sensor according to one embodiment.

FIG. 5 is an SEM image showing (a) before the tensile force is applied and (b, c) cracking along the crack lag after being applied.

FIG. 6 is an SEM image showing the manner in which cracks are formed at various gap lengths.

FIG. 7 is a graph (b, d) showing the difference (a) of the crack formation pattern at various gap lengths and the FEM simulation result (c) for identifying the difference.

FIG. 8 is a graph of the surface of a crack-based sensor formed disorderly without patterning and the resistance change measured using it.

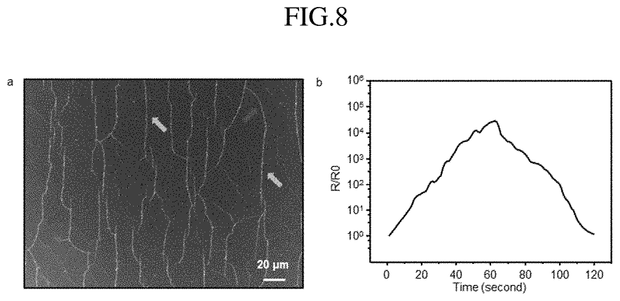

FIG. 9 is a conceptual diagram and a graph showing the change in resistance according to the tensile direction.

FIG. 10 is a load cell for measuring changes due to pressure and tension.

FIG. 11 is a graph showing changes in resistance due to loading and unloading according to a range of change rates and reproducibility through repeated experiments.

FIG. 12 shows the result of the resistance change measurement by loading and unloading according to the variation rate range and its hysteresis.

FIG. 13 shows a normalized resistance versus strain curve obtained by comparing the theoretical value according to Equation 6 with the experimental value measured by the crack sensor.

FIG. 14 is a graph showing the reaction rate for a sudden change.

FIG. 15 shows experimental results showing the change in resistance due to various pressing conditions of the pressure range (a) of 0 to 10 kPa, the pressure (b) by the small ant, and the pressure (c, d) by the wrist pulse.

FIG. 16 shows a high sensitivity sensor capable of simultaneously displaying a position and a pressure using a multi-pixel array, and a result of measurement using the same.

### BEST MODE

Hereinafter, the present invention is capable of various modifications and various embodiments, and specific embodiments are illustrated in the drawings and described in detail in the detailed description. It should be understood, however, that the present invention is not intended to be limited to any particular embodiment, but comprises all modifications, equivalents, or alternatives falling within the spirit and scope of the present invention. In the following description, a detailed description of related well-known arts will be omitted if it is determined that the gist of the present invention may be blurred. Recently, a mechanical crack based sensor using a non-crack parallel system with high sensitivity to deformation and vibration has been reported. However, in order to achieve ultrafast performance, sensitivity to force must be amplified by high stretchability and controllability through the formation of cracks.

The present invention provides an inexpensive, ultra-sensitive strain and pressure sensor based on induction of more accurate mechanical cracking within a regular microscale pattern.

The sensor according to the present invention can focus stress on a specific region around a hole by patterning a hole on the surface of the device, and can accurately form uniform cracks connecting the holes from this.

The sensor of the present invention is a sensor capable of measuring the tensile rate and measuring the pressure applied to the surface. It may be manufactured by generating mechanical cracks, after the metal thin film is deposited on the polymer. It may be effectively applied to wearable health care, and can replace existing extension sensors or pressure sensors.

Hereinafter, a high sensitivity sensor including a linearly induced conductive thin film containing cracks according to an embodiment of the present invention will be described in detail.

The present invention provides a high sensitivity sensor comprising:

a flexible support having a hole pattern formed therein; and

a conductive thin film formed on at least one surface of the support,

wherein the conductive thin film includes a linearly induced crack having a crack plane contacting at least a part of the surfaces and facing each other,

wherein the crack plane is linearly induced by a regular hole pattern formed on the flexible support,

wherein an external stimulus is measured by measurement of an electrical change caused by a change in contact area or a short circuit or re-contact while the crack plane moves according to an external physical stimulus.

Also, the present invention provides a manufacturing method of the high sensitivity sensor:

forming a regular hole pattern in the flexible support;

forming a conductive thin film on at least one surface of the flexible support; and

stretching the conductive thin film to induce a crack on a straight line.

The high sensitivity sensor according to the present invention can form a crack uniformly formed in a straight line along the hole pattern by the hole pattern formed on the flexible support and the formation of such a linearly formed crack can improve the sensitivity of the sensor.

The crack sensor according to the present invention is characterized in that when a conductive thin film formed on a hole pattern formed on the flexible support is subjected to external physical stimulation by tension or pressure, stress is concentrated around a position where the hole formed on the flexible support is located, thereby the cracks may be uniformly formed along the contact surface between the hole and the hole.

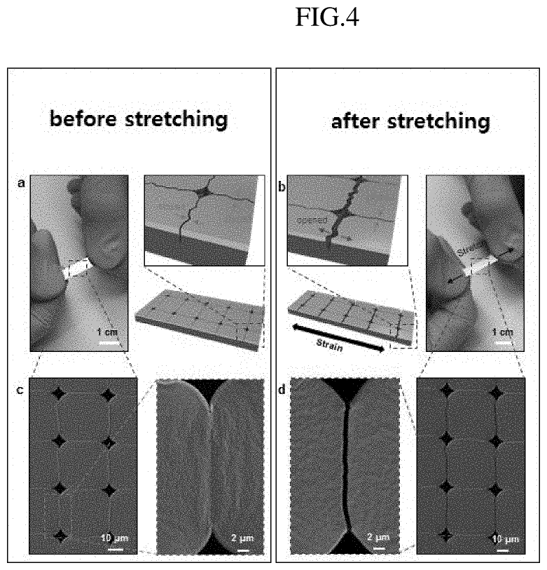

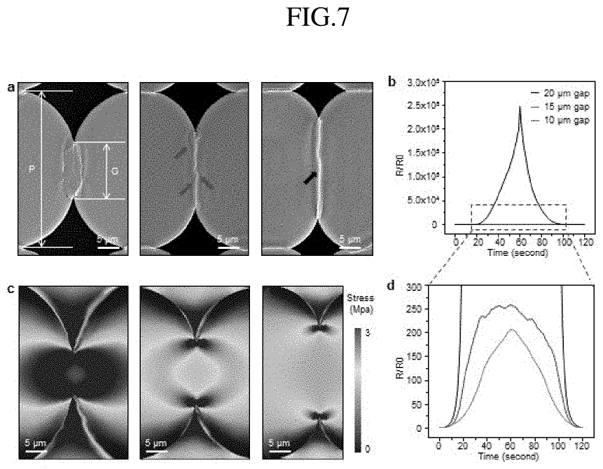

The crack plane is formed between the holes and the holes as shown in adjacent c and d of FIG. 4, and as shown in FIG. 7a, the length G of the crack plane may have a length of 50% or more, preferably 60% or more, of the length P of the straight line connecting the center of the hole adjacent to the center of the hole.

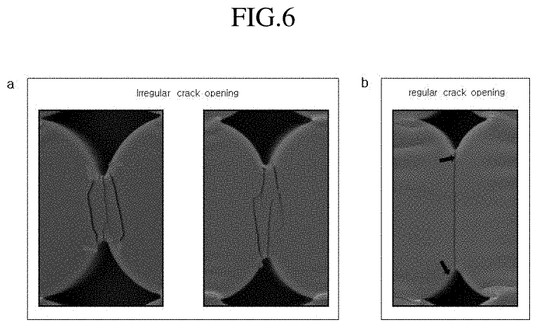

If the length of G is less than 50% of the length of P, cracks may not be formed in a straight line. As shown in FIGS. 6a and 7a, since several cracks may be formed in a shape other than a straight line, the sensitivity may be lowered.

According to one embodiment, the hole pattern may be any shape such as a circle, an ellipse, a rectangle, a diamond, a star, a cross, etc. preferably rhomboidal curves formed as shown in c and d of FIG. 4, i.e., a cross form or a curved rhombus having four vertices combined with four arcs may be suitable.

The above hole pattern provides directionality in generation of cracks at each vertex, thereby it may be advantageous to uniformly form a more straight-shaped crack.

As shown in FIGS. 4c and 4d, the crack sensor according to the present invention is generated by concentrating stress on two neighboring hole fans by an external force by a hole pattern, and cracks may be formed in a straight line along the hole pattern as shown in FIGS. 4d and 6b by external force.

When the tensile force is applied to a patterned crack, a crack formed perpendicularly to the axis of the force applied by the tensile force is opened, the cracks formed in parallel (horizontally) are closed.

As shown in FIG. 4c, by applying deformation by stretching to a tightly closed crack, the interval between cracks is enlarged as shown in FIG. 4d, so that the contact area between crack planes may be reduced, and this increases the electrical resistance. Since the disconnected crack plane has no conductivity, the resistance of the metal layer may be rapidly increased by the crack opened by the break.

Rarely, bridges and metal contacts between crack lips can lead to high strain sensitivity of resistance.

The high sensitivity sensor of the present invention can exhibit a sensitivity (ΔR/R₀) of 1 to 1×10⁶ at a strain of 0 to 10%.

The gauge factor of the high sensitivity sensor according to the present invention is defined as (ΔR/R₀)/E, and the gauge factor may be 2×10⁶ or more in a strain range of 0 to 10%.

The high sensitivity sensor according to the present invention can exhibit a sensitivity (ΔR/R₀) of 2×10⁴ or more at a pressure in the range of 7 to 10 kPa, and preferably, a sensitivity of 1×10⁵ or more at a pressure in the range of 8 to 9.5 kPa.

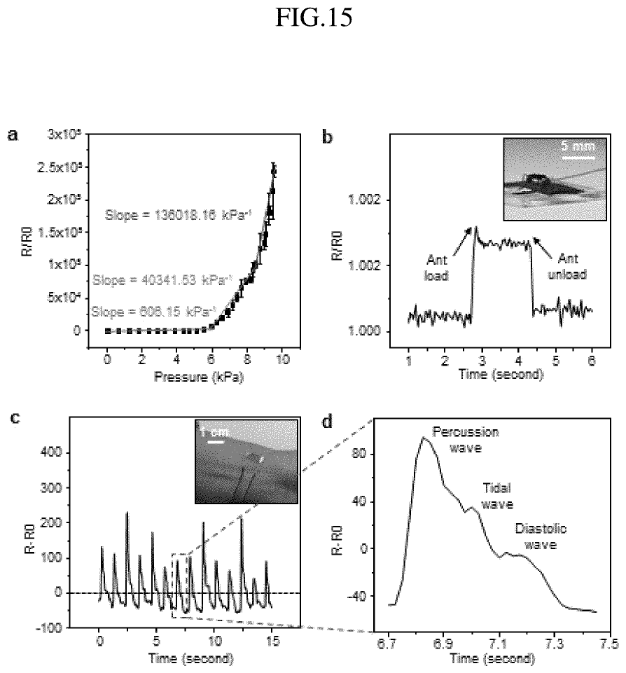

The present invention exhibits the high sensitivity due to pressure sensitivity, which may be used to measure a physiological signal such as a pulse by attaching it to the wrist as shown in FIGS. 15c and 15d. FIG. 15c is a result of measuring the pulse by attaching the high sensitivity sensor according to the present invention to the wrist, and FIG. 15d means that the high sensitivity sensor according to the present invention has high precision enough to distinguish the small differences in three steps of the pulsation such as percussion wave, tidal wave and diastolic wave.

According to one embodiment, the external physical stimulus may be applied at various angles to the crack plane, it can exhibit a better sensitivity when the axis of the force with respect to the direction in which force is exerted on the crack plane is applied at an angle of vertical (90°) or 45°, as the external physical stimulus. That is, when the external physical stimulus is applied symmetrically and uniformly to the shape of the hole pattern or the shape of the pattern of the conductive thin film formed by the cracks, the sensitivity may be greater. That is, the change in the gauge factor may be larger. More preferably, an external force may be applied to the crack plane in an angular range of 90°±10°.

The high-sensitivity sensor is a sensor for measuring external tensile or pressure by measuring a change in resistance of a conductive thin film as cracks formed on the conductive thin film are spaced apart according to tension or pressure.

That is, in a crack formed in the conductive thin film, there is a crack having a crack face in which at least some surfaces are in contact with each other while facing each other is present. When an external stimulus such as a tension or a pressure change is applied, the electrical resistance changes or an electrical short or open is formed as the contacted crack plane moves and the contact area changes, thereby a large change in the resistance value on the conductive thin film occurs. By detecting this, the conductive thin film structure may be utilized as a tensile sensor, a pressure sensor, and the like.

The conventional strain gauge sensor uses that the resistance increases as the metal thin film is stretched. However, the present invention uses that an crack gap in the metal thin film is spread. As the crack gap spreads, the electrical short circuit increases, and the resistance increases sharply. For the above reasons, the sensitivity is much higher than that of conventional strain gauge sensors.

According to one embodiment, the cracks present in the conductive thin film may be induced in a straight line according to a hole pattern formed on the flexible support, the degree of occurrence of the crack may also vary depending on the interval, shape, thickness of the conductive thin film, forming condition, and the like, and is not particularly limited.

In the high sensitivity sensor of the present invention, the flexible support may be any one or a combination thereof selected from a group consisting of polyurethane acrylate (PUA), polydimethylsiloxane (PDMS), polyethylene terephthalate (PET), polypropylene (PP), polyethylene (PE) and the like, and may be most preferably a polyurethane acrylate (PUA).

In the high sensitivity sensor of the present invention, it is preferable that the conductive thin film is any one or a combination thereof selected from the group consisting of Au, Ag, Pt, Cu, Cr and Pt, and may be most preferably Cr/Pt combination. According to one embodiment, the conductive thin film is not limited in its thickness, but it is preferable that the conductive thin film has a thickness enough to form a crack by a mechanical method such as tensile and bending. The conditions for forming such cracks may vary depending on the type of the conductive thin film and the flexible support.

In the high sensitivity sensor of the present invention, it is preferable that the thickness of the conductive thin film is 0.1 nm to 1 μm, more preferably 10 nm to 50 nm, even more preferably 20 nm to 30 nm. Also, the Young's modulus of the conductive thin film may be 10¹⁰ to 10¹².

In the high sensitivity sensor of the present invention, the gauge factor of the high sensitivity sensor may be 1×10⁵ to 1×10⁶ (1 to 10% tensile range). The gauge factor means the rate of change of resistance of the strain gauge to the occurring strain.

In the high sensitivity sensor of the present invention, the flexibility of the high sensitivity sensor means that it may bend to a minimum radius of 1 mm or more.

According to the above characteristics, the high sensitivity sensor of the present invention may be applied to various fields such as a pressure sensor, a tensile sensor, an artificial skin, etc., and may be used as a positioning detection sensor by pixelizing the sensor.

In the present invention a theoretical analysis of resistance versus strain data is performed the theoretical analysis result is consistent with the result of experimental data in deformations that were not too large.

The present inventors have found a universal mechanism for a strain sensor based on a parallel crack formed on a uniform 20 nm Pt film that is cracked on a polymer having a low elasticity. In the sensor, the free-crack cuts sensor strip by a technique of producing a large unidirectional strain. The normalization conductance S vs strain rate E of the sensor, the following Equation (1), is defined by the probability distribution function of the steps on the crack lips forming the contact between the crack lip, P (x).

S=∫_{ε}^{∞}P(x)dx (1)

For free cracks, the expression P (x) has only parameters related to size. The strain Co corresponds to the width kε₀ of the crack gap, and kε₀ is the grain size x₀=kε₀.

P(x)=P(1/x)/x² (2)

In the above Equation, x=ε/ε₀ and k is a proportional coefficient defined in relation to the crack gap width with respect to the strain rate. k may be different depending on the material constituting the parallel crack system, which may be obtained from the experiment.

Physically, Equation 2 indicates that the small step of the cracked body formed by the shift of the grain is the same distribution as the large step formed by the accumulation of the grain, This may not be able to distinguish large and small meandering protrusions because there is a scale and an elastic region of the substrate that does not have any length characteristics.

One solution to Equation 2 is to select a log-normal pdf

$$P ( ɛ ) = \frac{1}{ɛμ \sqrt{π}} exp ( - \frac{{( ln ( ɛ \text{/} {ɛ}_{0} ) )}^{2}}{{μ}^{2}} ) \qquad ( 3 )$$

or almost identical log-logistic pdf

$$P ( ɛ ) = \frac{B}{{ɛ}_{0}} \frac{{( ɛ \text{/} {ɛ}_{0} )}^{B - 1}}{{( 1 + {( ɛ \text{/} {ɛ}_{0} )}^{B} )}^{2}} \qquad ( 4 )$$

In the above Equations, μ and B are variables of pdf.

The distribution of Equation 3 and the distribution of Equation 4 all belong to the classification of an asymmetric distribution having a so-called long tail.

The non-zero probability of formation, except for rare contact between crack lip, is in the nature of the conduction mechanism through cracks and therefore coincides with the long tail distribution.

Equation 3 and Equation 1 provide the resistance R=1/S as a function of strain as follows:

$$R = 2 \text{/} ( 1 - erf ( \frac{ln \text{?} )}{\text{?}} ) ) \\ \text{?} \text{indicates text missing or illegible when filed} \qquad ( 5 )$$

erf (x) is an error function. Equation 5 renders a normalized resistor. The normalized resistance is remarkably consistent with experimental results with strain rate of up to 2%.

At the same time, the log-logistic pdf of Equation 4 may be derived from Equation 1 together with the following Equation.

R=1+(ε/ε₀)^{B} (6)

The above Equation is consistent with the experiments with fitting parameters ε₀=0.39 and B=2.39, it has the same precision as the log-normal pdf of Equation 5.

However, the power-law function of Equation 6 is much simpler than the error function of Equation 5.

The present invention can propose a universal exponential rule for data fitting by an experimenter who is studying free parallel cracks.

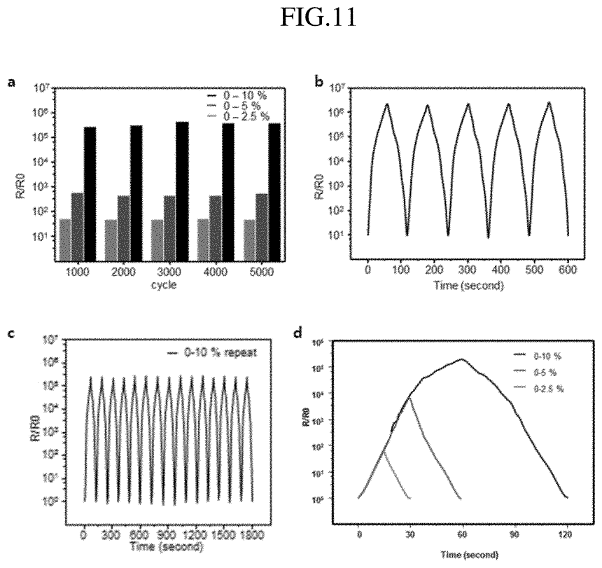

Surprisingly, after changing the uniform Pt film strip to a patterned strip on a much more stretchable polymer (FIG. 4a), the strain rate dependence of the resistance has changed tremendously from the exponential law of Equation 6 to the exponential function over a wide range of strain rates over 5%. FIG. 11d shows that a linear pattern appears in the semi-log plot.

Here, the basic mechanism of this phenomenon is explained.

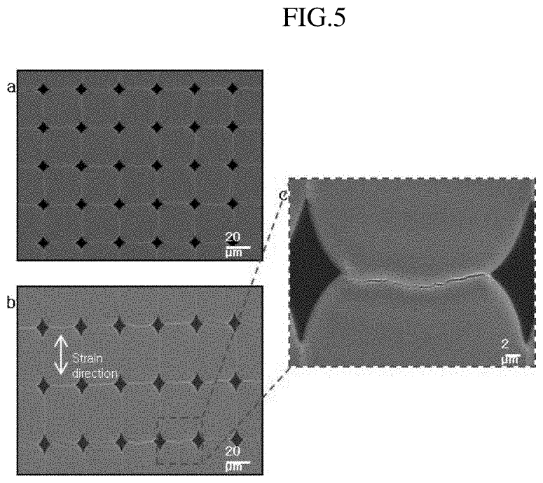

Important differences of the cracks formed in the present invention and the previous study are shown in FIGS. 5b and 5c.

The cracks between the pattern and the patches closely follow “crest” of wrinkles on the metal/polymer film.

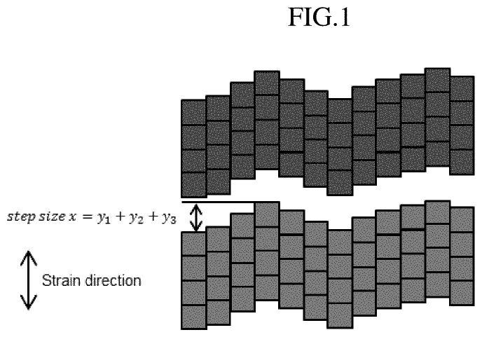

That is, this means that the crack passage is very direct, and only the neighboring platinum particles are separated along the crack lips (FIG. 1).

In this regard, the local deviation is related to the size of the grain, and therefore may not satisfy Alteration Equation 1 for free crack creation.

On the other hand, as shown in FIG. 5b, the pattern patches are pressed against each other in the horizontal and vertical directions in the deformation direction, this is due to Poisson's ratio of 0.5, which is the intrinsic property of rubber-like material. Thus, the system is virtually unchanged on a one-dimensional with a cut through a crack located in a current set of horizontally aligned rectangles (referring to FIG. 5).

Similar to this study, it is enough to calculate the step pdf.

According to FIG. 1, each i-th particle along a crack (crack trajectory) lip can move up and down with a probability of ½ and a yi shift (in the direction of deformation).

Crack step size refers to the distance traveled by the upward (downward) trajectory by several adjacent particles.

As shown in FIG. 1, for example, the sum of three grain movements in one direction produces an X-sized step. Suppose that the local grain shift is distributed with local pdf P (y).

The grains adjacent to the normalized size 1 moving vertically to the small steps of y1, . . . , y2 may have an overall pdf P (x) function of step size x.

$$P ( x ) = \int \int {\int }_{0}^{1} {\sum }_{n} δ ( {y}_{1} + ⋯ + {y}_{n} - x ) \frac{1}{{2}^{n}} P ( {y}_{1} ) … P ( {y}_{n} ) {dy}_{1} … {dy}_{n} \qquad ( 7 )$$

In this equation,

∫₀¹P(y)dy=1 (8)

δ is a delta function, and n=1, 2, . . . , . The delta function represents the fine pdf of the step consisting of the movement of a positive value n in the direction satisfying the expression y1+ . . . +yn−x=0.

According to the above equation, the probability of up-and-down movement of particles is ½, as assumed.

Thus, if defining a step as a total upward movement, the probability of a given configuration for the small steps of n is proportional to ½ⁿ.

Then, by rewriting Equation 7 with a Fourier integral of the delta function,

$$P ( x ) = 1 \text{/} 2 π {\int }_{- ∞}^{∞} \int \int {\int }_{0}^{1} {\sum }_{n} exp ( ia ( {y}_{1} + ⋯ + {y}_{n} - x ) ) \frac{1}{{2}^{n}} P ( {y}_{1} ) … P ( {y}_{n} ) {dy}_{1} … {dy}_{n} da \qquad ( 9 a )$$

Alternatively, Equation 9a may be simplified as follows for independent integration through each yi

P(x)=½π∫_{-∞}^{∞}Σₙ exp(−iαx)[½f(α)]ⁿdα (9)

In this equation,

f(α)=∫₀¹P(y)exp(iαy)dy (10)

The geometric series of Equation 9 may be directly transformed into Equation 11 below.

$$P ( x ) = 1 \text{/} 2 π {\int }_{- ∞}^{∞} exp ( - iax ) \frac{\frac{1}{2} f ( a )}{1 - \frac{1}{2} f ( a )} da \qquad ( 11 )$$

The Cauchy integral of Equation 11 may be analyzed in general terms.

It may be shown that the collapse of function P (x) at a large value of x may be nearly exponential and almost independent of the particular form of P (y).

P(x)˜exp(−z₀x) [symbol] x>>1 and z₀>0 (12)

If one pole plays a dominant role in the denominator of Equation 11,

1−½f(−iz₀)=₀ (13)

The lowest actual value z₀>0 in Equation 13.

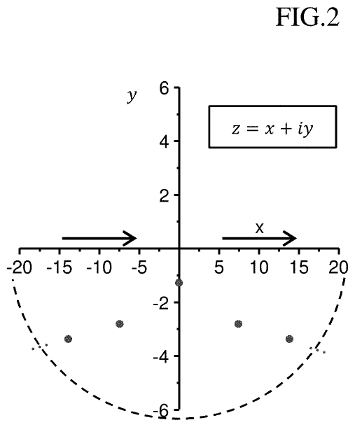

All other poles (all solutions of Equation 13) may be complex and may be placed at the bottom of the complex plane (see the example of FIG. 2).

Equation 10 shows that a single pure imaginary pole, α=−iz₀, always exists, otherwise it is impossible for the integral of Equation 10 to be equal to 2 when having a pole at the upper half.

Indeed, if α=−iz₀ and |exp(iαy)|=|exp(−z₀y)|≤1, and if it is impossible to integrate f(α) Of Equation 10 with a value greater than 1, this is because the integral includes a normalized probability function, for all y in Equation 10, even if |exp (iαy)| is exactly 1, the maximum value is only given as 1.

However, Equation 13 is not satisfied because f(α)=2>1.

Conveniently, P(x) is obtained as the sum of the rest of the poles by closing the Cauchy integral shape of Equation 11 by an infinitely large half circle in the bottom plane (FIG. 2), and the largest value of the exponent term dominated by pole −iz₀ will appear predominant in large x. If it is limited itself by this pole, a normalized probability may be obtained.

P(x)=exp(−z₀x)z₀ (14)

It may be seen from Equation 1 that conductance S is not only at a high strain rate,

S=∫_{ε}^{∞} exp(−z₀x)z₀dx=exp(−z₀ε) (15)

It is also an exponential function of the strain due to the resistance.

R=1/S=exp(z₀ε)≡exp(ε/ε₀) (16)

The power-law function and the exponential function are the differences between Equation 6 and Equation 16.

Considering the most common example of P(y)=1 assuming the position of any grains adjacent to each other, a uniform distribution of grains is moved along the crack lip in FIG. 1. In this case, Equation 10 improves Equation 17 below,

f(α)=(exp(iα)−1)/iα (17)

Then, Equation 13 has the following form.

2z₀+1−exp(z₀)=0 (18)

The solution of Equation (17) may be confirmed numerically. The lowest z₀=1.256, and the other poles are 2.789±7.438i, 3.360±13.866i . . . (see FIG. 2).

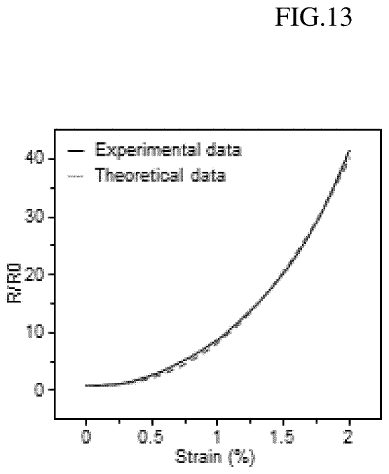

FIG. 13 provides a strain rate (red line) calculated with a normalized resistance vs P(y)=1 along with a pure exponential function of Equation 16 (black line) to see the agreement between the experimental data and the theory do. On the other hand, in the asymptotic Equation 16, the strain should be rescaled by α=7 times, such that, for example, it should be consistent with the resistance vs strain calculated by the uniform pdf of the grains, the linear slope of the experiment in FIG. 11d.

Physically it means that the movement of the grains is limited to 30%, thus the crack lips is planarized.

Therefore, the resistance reacts as if it were planarized by increasing the slope of the resistance at the semi-logarithmic scale.

The parameters measure the flatness of the crack lips.

From FIG. 1, it may be seen that the maximum slope of the step projection is limited by α, α is the tangent of the maximum slant angle.

The maximum tilt angle at P(y)=1 is 45 degrees (°) which is tangent α=1.

Of course, if the crack lips are perfectly flat with α=0 without any movement, they can have a sudden separation of cracks and an infinite slope of R/R₀.

According to the fitting of FIG. 2e, the parameter of the strain measured in % is

$${ɛ}_{0} = \frac{a}{{z}_{0}} = \frac{0.7}{{2}_{0}} \approx 0.6 .$$

With this close approximation, a specific grain size x₀ is able to be calculated.

According to the SEM image, the distance x of the gap is proportional to the strain x=kε, in this case, k≈50 nm with c in %; the particle size x₀=kε₀=30 nm may be very close to the primary particle size component of the granulated Pt film.

### MODE FOR INVENTION

Hereinafter, embodiments of the present invention will be described in order to facilitate understanding of the present invention, it will be apparent to those skilled in the art that various modifications and variations can be made in the present invention without departing from the spirit and scope of the invention as defined by the appended claims. It is obvious that such variations and modifications fall within the scope of the appended claims.

### <Embodiment 1> Fabrication of High Sensitivity Sensor Based on Induction Crack

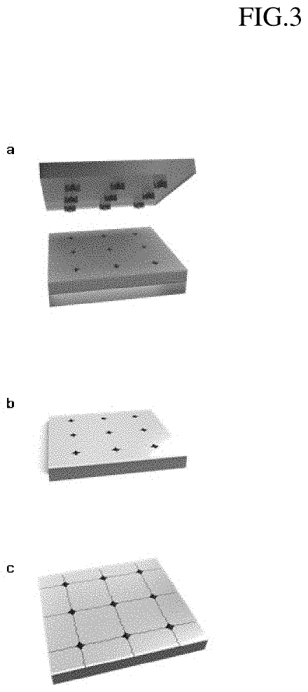

Crack sensors were fabricated as shown in FIGS. 3a-3c.

Specifically, 100 μm polydimethylsiloxane (PDMS) coated with a spin coating was treated with an oxygen plasma using a plasma surface processor CUTE-1 MPR (Femto Science Inc.) and bonded on glass. After 20 μl of polyurethane acrylate (PUA) was dropped on the PDMS/glass mold, the filler-patterned silicone mold was covered and irradiated with 350 nm UV (about 12 mJ/cm²). The patterned 10 nm chromium layer was formed by thermal evaporation by a thermal evaporator (Selcos Inc.), and a sputtered 20 nm platinum layer was deposited. The metal layer deposited PUA film was carefully removed from the PDMA/glass mold and then stretched 5% in the x/y direction using a customized stretcher. The crack sensor before and after stretch is shown in FIG. 4.

The wires were then attached using a conductive polymer so that electrical signals could be connected to the sensor. The high-sensitivity sensor thus manufactured is shown in FIGS. 4 and 5. FIGS. 4 and 5 show that cracks are widen as the deformation is applied to the high-sensitivity sensor.

### <Embodiment 2> Multi-Pixel Array Sample Manufacturing

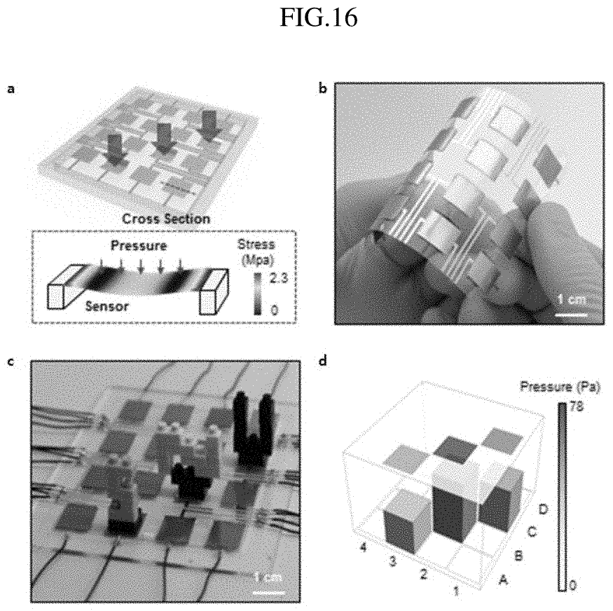

To demonstrate the scalability and capability of the device for detecting mechanical vibrations and pressures, a sensor network of 16 pixels (4×4 pixel array) is provided on an area of 6×6 cm² as shown in FIG. 16b. Schematic views of a multi-pixel system are shown in FIGS. 16a and 16c. Each pixel (1×1 cm² islands) was constructed with a thickness of 100 μm PUA/10 nm Cr/20 nm Pt with a hole pattern and then elongated and stretched 10% bi-axially to generate cracks. The electrical connection between the cracked Pt and the Lab View-based PXI-4071 system (NI instrument Inc.) was formed by a gold line (Au, 50 nm thick) deposited on a PET film using the shadow mark scheme. Each manufactured pixel was placed on a PET film by a conductive polymer (CW2400, circuitworks) in an independent manner or electrically connected by a gold line.

### <Experimental Example 1> Measurement of Change in Resistance (Gauge Factor) According to Length of Crack Gap

In order to confirm the effect of the linear crack using the high sensitivity sensor of Embodiment 1, cracks were formed by using three different hole patterns.

As shown in FIG. 7a, P is the shortest distance from the center of the hole and is the same in all three patterns tested, G is the length of the gap, and the gap represents the shortest distance between the tips of the holes.

When the lengths of G are 10 μm, 15 μm and 20 μm, crack formation patterns and resistance changes are shown in FIGS. 6a and 6b and FIGS. 7a to 7d.

FIG. 6a shows that several cracks may be induced when the length of the gap G is 10 μm and 15 μm, and 6b shows that a very straight crack may be generated when G is 20 μm.

The occurrence of a number of non-intact cracks as shown in FIG. 6a can reduce the sensitivity to changes in resistance, and these results are shown in FIGS. 7b and 7d.

To understand this nonuniformity, we performed a finite element method (FEM) simulation and the simulation results are shown in FIG. 7c. The results in FIG. 7c show that narrow pattern gaps create a wider distribution of high stresses that stimulate everywhere cracks appear in the gap distance. In addition, if the gap length G does not have a sufficient length for P, the section where the stress acts on the crack surface where cracks occur may be too wide, from this, it is possible to induce cracks by generating stress at various points.

The crack of the crack sensor according to the present invention is advantageous when it is guided in a straight line, which is shown in the results of FIGS. 7b and 7d.

In addition, the resistance change at 20 μm in FIG. 7b shows a sharper graph compared to a sensor based on disordered cracks, and this indicates a change in resistance according to a change in the distance of the crack lip, and that a linear crack responds more accurately to the change in the distance of the crack.

### <Experimental Example 2> Measurement of Change in Resistance (Gauge Factor) According to Stretching Angle

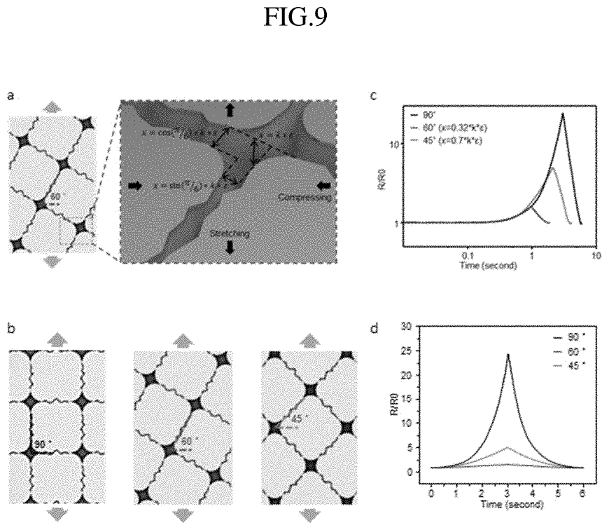

To demonstrate the possibility of a theoretical concept of resistance depending on a single parameter (normalized gap size x/x₀=kε/x₀) of the high sensitivity sensor prepared in Embodiment 1, for the normalized resistance vs. strain, we placed the square pattern at 60 to 45° as shown in FIG. 9b to compare them to the case of 90°. The experimental results are shown in FIGS. 9c and 9d.

By reflowing to the log-log scale, a curve of 60 degrees (°) corresponds to a 90 degree (°) curve after adjusting the strain to 0.32 (see FIG. 9c).

Geometrically 90−60=30, this is because the gap size is provided by deformation from x=kε to sin(π/6)x=k(0.5ε) or to k(0.32ε) or more due to additional shrinkage due to deformation in the orthogonal direction of the sample, it is explained by effectively narrowing the proper gap size (FIG. 9a).

The difference in the complementary angle of 60° may be somewhat related here because the conductance is governed by most of the conduction pathways that occur through the narrow gap at 30°.

In the case of 45° at the same angle, the rescaling factor is 0.7, and therefore, sin(π/4)=1/√2 (FIG. 9c).

FIG. 9d shows the result of a change in the resistance of the crack generated in the lattice form as the angle changes, when the angle is 90°, the greatest change in resistance appears, and the resistance changes in the order of 45° and 60°.

Therefore, the resistance change is more sensitive than when the rectangular patch formed by the cracks generated in the lattice form is stretched through the force symmetrical at the same complementary angle, the crack distance may be more effectively widen, the complementary angle according to the difference of (90°—stretching angle) is 45° or less at an angle of 45° or more, and from this, it may be indicative of a lower resistivity compared to 45° as they form a narrower crack-to-crack distance. However, this may be less affected at an angle close to 90°.

### <Experimental Example 3> Measurement of Resistance Change According to Strain Change

The resistance of the high-sensitivity sensor of Embodiment 1 was measured by applying a current while applying tension thereto. Specifically, FIGS. 11a to 11d show the change in electrical resistance measured after stretching up to 10% and then again to the original state, i.e., 0% strain state, and FIGS. 11a to 11c are graphs showing hysteresis and reproducibility of the sensor according to Embodiment 1.

The high sensitivity crack sensor of Embodiment 1 was fixed by customized pressure test equipment.

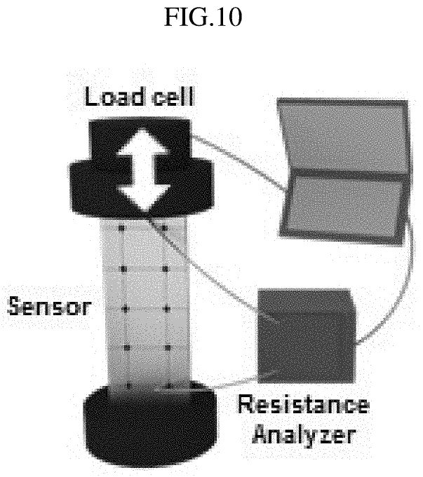

Continuous pressure was applied to a crack sensor constructed on the basis of Lab VIEW (NI instrument) based on PXI-4071 resistance analyzer (NI instrument) and the load cell (2712-041, Instron Co.) of FIG. 10.

FIG. 11a shows the results of the reproducibility test measured in 5000 repeated cycles in a strain rate range of 0 to 2.5%, 0 to 5% and 0 to 10%, FIG. 11b shows reproducibility after 5000 cycles or more in the range of strain of 10%. From these, it may be seen that the crack sensor according to the present invention shows almost no difference in performance even after repeated measurement of 5,000 times or more.

FIG. 11c shows the reproducibility result obtained by repeating the loading-unloading test using the loading cell of FIG. 10, 1800 times in the strain rate range of 0 to 10%, and from the results of the graph, it may be seen that the crack sensor according to the present invention has excellent reproducibility.

As shown in FIG. 11d, when measuring the electric resistance after stretching the sensor of Embodiment 1 up to 10% and back to the original state, that is, the state of 0% strain, it was found that the change of electric resistance was changed up to about 2×10⁵ times of the initial resistance, Repeatedly the same type of resistance change was obtained reproducibly. This is because the contact area decreases as the strain is applied to the crack plane which is in contact with each other, finally it becomes separated, and thereby the electrical resistance sharply increases. And, as the strain is removed, the sensor shrinks and the spaced crack surface comes into contact, and as the contact area increases, it returns to its original state.

### <Experimental Example 4> Measurement of Resistance Change According to Strain Change

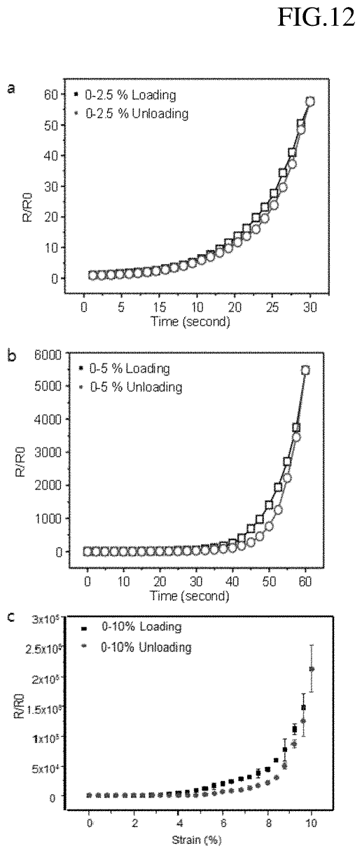

FIGS. 12a to 12c show graphs of measured resistance changes measured in loading and unloading tests in a strain range of 0 to 2.5%, 0 to 5%, and 0 to 10%.

From the results of FIGS. 12a to 12c, the crack-based sensor of Embodiment 1 while according to the present invention shows little hysteresis in the loading and unloading process, as the range of applied strain increases, the hysteresis increases somewhat.

FIG. 12c shows graphs with the standard deviation using the average value of the values measured in five samples.

The present invention has performed a theoretical analysis of strain data with respect to resistances (Equations 1-18). FIG. 13 shows plots of strain-resistance change curves fitted on the basis of experimental versus theoretically obtained data with respect to resistance variation with strain rate. From the above results, it may be seen that the crack sensor according to the present invention exhibits a pattern almost coinciding with the result of experimental data in a strain range that is not too large.

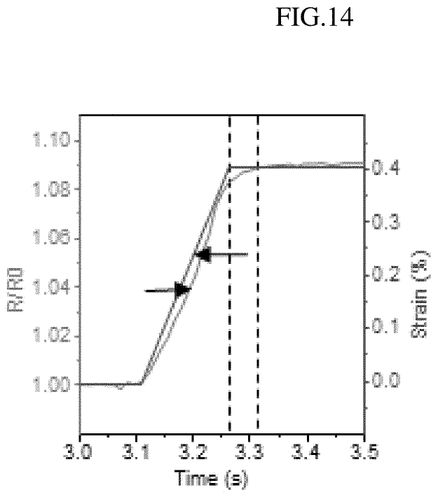

FIG. 14 is a graph showing the reaction time when a sudden change is given, the results of the experiment show that the reaction occurs within 100 ms. It may be seen from FIG. 14 that the change aspect in the strain rate and the change aspect in resistance show almost the same reaction pattern.

### <Experimental Example 5> Measurement of Resistance Change According to Pressure

Applying the pressure can stretch the sample and increase the resistance of the metal film. For the measurement of the pressure, the crack-based sensor of Embodiment 1 was mounted on a customized machine, and resistance data may be measured with a resistance analyzer (PXI-4071, National Instruments).

Pressure data was obtained with load cell 2712-041 (Instron Co.) in FIG. 10. The resistance of the obtained pressure data may be linearized into three pressure regions, and this is shown in FIG. 15a. The graph of FIG. 15a shows the three pressure area:

1) slope at 0-6 kPa 606.15 kPa⁻¹;

2) slope at 6-8 kPa 40341.53 kPa⁻¹;
3) slope at 8-9.5 kPa 136018.16 kPa⁻¹.

The slope of this pressure-resistance curve exhibits significantly better sensitivity than 192 kPa⁻¹ at pressures in the range of 0-5 kPa, the highest performance of the pressure sensitivity represented in the reported studies (Y. Zang. et al. Flexible suspended gate organic thin-film transistors for ultra-sensitive pressure detection. NATURE COMMUNICATIONS, 6:6269, doi: 10.1038/ncomms7269).

FIG. 15b shows a result of measuring a small antic mass (Ponera japonica, 1 mg) corresponding to a pressure of 0.2 Pa using the crack sensor, and this is a result indicating that the crack sensor according to the present invention exhibits high sensitivity to pressure.

The crack sensor was mounted on the wrist to measure the physiological signals of the wrist pulse.

FIGS. 15b and 15d are graphs showing physiological signals of the wrist pulse, FIG. 15d shows a result of enlarging a part of the graph 15c. From the graph of FIG. 15d, it may be seen that the crack sensor according to the present invention exhibits a sensitivity high enough to measure all the minute three-step changes of the wrist pulse.

### <Experiment 6> Measurement of Position and Pressure Through a High-Sensitivity Sensor Array

To demonstrate sensor scalability and spatial resolution and pressure sensing capabilities, a multi-pixel array was fabricated by the method of Embodiment 2, which is shown in FIG. 16a. The crack-based device is highly flexible, and there may be a warp as shown in FIG. 16b.

The small LEGO-form slices S, N, U were carefully positioned in the sensor of Embodiment 2 with the array of pixels as shown in FIG. 16c, and the pressure and position derived therefrom could be easily detected from the array sensor. The results measured from the array sensor are shown in FIG. 16d.

While the present invention has been particularly shown and described with reference to specific embodiments thereof, those skilled in the art will appreciate that such specific embodiments are merely preferred embodiments. It will be apparent that the scope of the present invention is not limited thereby. Accordingly, the actual scope of the present invention will be defined by the appended claims and their equivalents.

1. A high-sensitivity sensor, comprising:
a flexible support having a hole pattern formed therein; and
a conductive thin film formed on at least one surface of the support,
wherein the conductive thin film includes a linearly induced crack having a crack plane contacting at least a part of the surfaces and facing each other,
wherein the crack plane is linearly induced by a regular hole pattern formed on the flexible support,
wherein the high sensitivity sensor measures an external stimulus by measurement of an electrical change caused by a change in contact area or a short circuit or re-contact while the crack plane moves according to an external physical stimulus.

2. The high-sensitivity sensor according to claim 1, wherein on the crack plane, a stress due to an external force is concentrated between adjacent holes, and thereby a crack is induced in a straight line along the hole pattern.

3. The high-sensitivity sensor according to claim 1, wherein the crack plane is provided between adjacent holes and the length G of the crack plane has a length of 60% or more with respect to the straight line P connecting the centers of adjacent holes where the crack plane is located.

4. The high-sensitivity sensor according to claim 1, wherein an angle of an external force applied to the crack plane is applied in a direction forming 90° or 45° with respect to the crack plane.

5. The high-sensitivity sensor according to claim 1 having a sensitivity of 2×10⁴ or more, at a pressure in the range of 7 to 10 kPa.

6. The high-sensitivity sensor according to claim 1, wherein the shape of the hole pattern is a rhombic shape made of a curved line or a cross shape having four vertices coupled with four arcs.

7. The high-sensitivity sensor according to claim 1, wherein the flexible support may be any one or a combination thereof selected from a group consisting of polyurethane acrylate (PUA), polydimethylsiloxane (PDMS), polyethylene terephthalate (PET), polypropylene (PP), and polyethylene (PE).

8. The high-sensitivity sensor according to claim 1, wherein the conductive thin film may be any one or a combination thereof selected from the group consisting of Au, Ag, Pt, Cu, Cr, Pt and the like.

9. The high-sensitivity sensor according to claim 1, wherein the crack is a nano-level fine crack.

10. The high-sensitivity sensor according to claim 1, characterized by electrical shorting or opening of the crack occurs by external stimulation, thereby changing the electrical resistance value of the conductive thin film.

11. The high-sensitivity sensor according to claim 1, wherein the external stimulus is either stretch or pressure or a combination thereof.

12. The high-sensitivity sensor according to claim 1, wherein the conductive thin film has a thickness of 0.1 nm to 1 μm.

13. The high-sensitivity sensor according to claim 1, wherein a gauge factor of 1 to 2×10⁶, at a strain rate of 0 to 10%.

14. The high-sensitivity sensor according to claim 1, wherein the flexibility of the high sensitivity sensor is able to be bent to a minimum radius of 1 mm or more.

15. A pressure sensor comprising the high-sensitivity sensor according to claim 1.

16. A strain sensor comprising the high-sensitivity sensor according to claim 1.

17. Pressure and strain sensors comprising the high-sensitivity sensor according to claim 1.

18. An artificial skin comprising the high-sensitivity sensor according to claim 1.

19. A method for manufacturing the high sensitivity sensor according to claim 1, comprising:
forming a regular hole pattern in the flexible support;
forming a conductive thin film on at least one surface of the flexible support; and
stretching the conductive thin film to induce a crack on a straight line.
